(19) United States
(12) Patent Application Publication
(10) Pub. No.: US 2011/0229551 A1
(43) Pub. Date: Sep. 22, 2011

(54) DRUG DELIVERY COMPOSITIONS AND METHODS USING NANOFIBER WEBS

(75) Inventors: JAYESH N. DOSHI, CHATTANOOGA, TN (US); LARRY RAYMOND DEBARGE, CHATTANOOGA, TN (US)

(73) Assignee: NOTUS LABORATORIES, INC., CHATTANOOGA, TN (US)

(21) Appl. No.: 12/725,569

(22) Filed: Mar. 17, 2010

(51) Int. Cl. A61K 9/70; A61K 31/616; A61K 31/573; A61K 39/395; A61P 29/00; A61P 31/00; A61P 27/06; A61K 38/22

(52) U.S. Cl. 424/443; 514/165; 514/180; 424/141.1; 977/906; 514/7.6

(57) ABSTRACT

Polymeric nanofibers have been developed which are useful in a variety of medical and other applications, such as filtration devices, medical prostheses, scaffolds for tissue engineering, wound dressings, controlled drug delivery systems, cosmetic skin masks, and protective clothing. These can be formed of any of a variety of different polymers, both non-biodegradable or biodegradable, and derived from synthetic or natural sources.

The present invention discloses 1) the composition of fibrous articles and 2) methods for using these articles in medical applications.

The biodegradable fibrous articles, which are preferably formed by electrospinning polymer solution of biodegradable fiberizable material with or in conjunction with medicinal agents and bioactive materials, comprise a composite (or asymmetric composite) of nanofibers with actives.

Nanofibrous articles having specific medical uses include controlled drug delivery devices, glaucoma implants, tissue engineering, wound dressings, reinforcement grafts, corneal shields, and orbital blowout or sinus reconstructive materials.

The methods include controlled drug delivery of a medicinal agent and providing treatment for inflammation, infection, trauma, glaucoma, and degenerative diseases.

The drug delivery compositions and methods of this invention are directed towards improving the delivery of drugs to a target area of the body. These drug delivery compositions are nanofiber webs, mats, or whiskers which incorporate an active ingredient for delivery into a bodily fluid. The active ingredient is delivered in a controlled manner by placing the nanofiber web into the bodily fluid which allows the drug embedded in the nanofiber to be released in a controlled and longer lasting manner.

### FIELD OF THE INVENTION

This invention relates to drug delivery compositions and methods, more particularly to drug delivery using nanofiber webs.

### BACKGROUND OF THE INVENTION

Therapeutic options for treatment of severe uveitis (ocular inflammatory disease) usually have been limited to topical and systemic corticosteroids and oral immunomodulators such as methotrexate. These systemic medications carry risks of severe complications, including death. Depot injections of long-lasting corticosteroids have been placed around the eye since the 1960's, but can result in scarring and decreased absorption with repetitive use. Injections of agents directly into the vitreous cavity of the eye are being done with increasing frequency, but require retreatment. For example, antiviral agents against cytomegalovirus, the blinding scourge at the height of the AIDS epidemic, were placed directly into the vitreous cavity at weekly intervals to patients intolerant of the side effects of near constant intravenous therapy. Intravitreal corticosteroids, primarily triamcinolone, are being more commonly performed for severe non-infectious uveitis, but have limited duration of action, vehicle toxicity issues, and require re-treatment.

Potential consequences of intravitreal injections include infection (endophthalmitis), hemorrhage, retinal detachment, and lens damage, not to mention the psychological issues of sticking sharp objects into the eye. Hence the need for improved local therapy.

A biodegradable, sustained-released drug delivery device (DDD) has the benefits of 1) delivering the active agent exactly where it is needed, limiting the untoward side effects for the rest of the body, 2) higher concentrations of active agent, 3) longer therapeutic interval, 4) fewer re-treatments, and 5) eliminates the need to remove and replace the spent device. Current options include conventional corticosteroid intravitreal implants: a recently FDA-approved non-biodegradable implant developed by Psivida, Inc. (Retisert™), and two intravitreal implants in phase 3 FDA testing (Osurdex™, Allergan Pharmaceuticals and Medidur™, also licensed by Psivida to Alimera Sciences).

The Retisert™ requires invasive surgery (with attendant risks of hemorrhage, infection, vitreous loss, retinal detachment), an excessive duration of activity (30 months), need for removal and replacement of the spent device, and local side effects of cataract formation (nearly 100% during treatment) and glaucoma (˜30%) due to the fluocinolone corticosteroid.

The Osurdex™ implant is a biodegradable pellet using poly(lactide-co-glycolide) acid (PLGA) as the biodegradable polymer vehicle and the conventional corticosteroid dexamethasone. The Medidur™ implant is non-biodegradable tubular implant device which delivers dexamethasone, and is left inside the eye after the device is spent.

Current needs include a bioerodable, sustained release DDD which can deliver not only corticosteroids, but sustained release antibiotics (e.g., sulfa drugs for toxoplasmosis), anti-parasitic agents (toxocariasis), and biological agents (e.g., anti-vascular endothelial growth factor monoclonal antibodies for neovascularization). Uveitis is relatively uncommon, yet causes 10% of the blindness in the U.S. According to Bausch &Lomb, posterior uveitis is the third leading cause of blindness in the U.S., affecting 175,000 people and an estimated 800,000 people worldwide.

Aside from the orphan drug applications for uveitis, there is a huge need to treat the exudative maculopathies common to macular degeneration, diabetic retinopathy, and retinal vascular disorders. Current therapy includes monthly injections of aptamers or recombinant humanized monoclonal antibodies, such as ranibizumab and bevicuzumab. Diabetic retinopathy and macular degeneration are the two leading causes of blindness in adults in the western world. There is presently no sustained release device for these biological agents, although phase 3 testing is underway to use the Osurdex™ implant for diabetic macular edema.

Therefore there is a pressing need for an improved drug delivery system especially for the eye. The drug delivery compositions and methods of this invention are directed towards this goal. These drug delivery compositions are nanofiber webs which incorporate an active ingredient and a bodily fluid. The active ingredient is delivered in a controlled manner by placing the nanofiber web into the bodily fluid which allows the drug embedded in the nanofiber to be released in a controlled and longer lasting manner.

### BRIEF SUMMARY OF THE INVENTION

One aspect of the present invention is drug delivery compositions containing a nanofiber web, impregnated with an active ingredient which is introduced into a bodily fluid.

In another aspect of the present invention, is a method of drug delivery by placing or positioning a nanofiber web containing an active ingredient into a bodily fluid.

### BRIEF DESCRIPTION OF THE DRAWINGS

FIG. 1 is a schematic representation of electrospinning process.

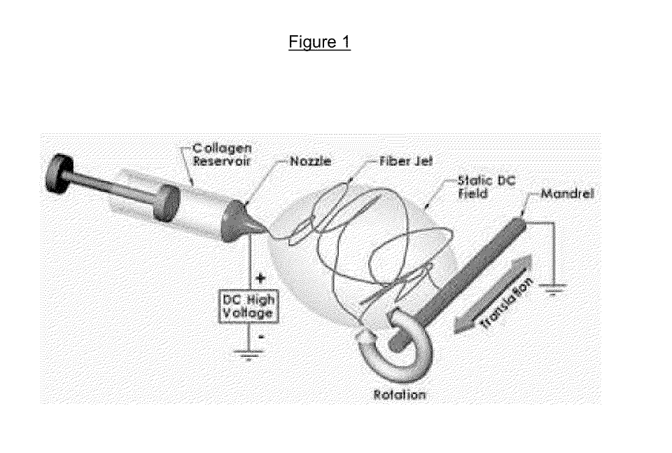

FIG. 2 is a scanning electron micrograph of PLGA nanofibers impregnated with triamcinolone acetonide.

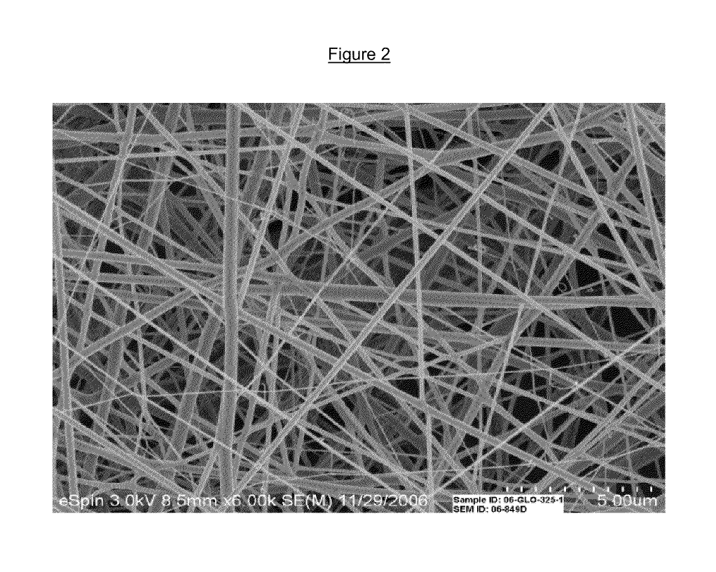

### DETAILED DESCRIPTION OF THE INVENTION

The drug delivery composition of this invention is a nonwoven nanofiber web or mat containing an active ingredient or ingredients. Preferably the active ingredient is dispersed throughout a matrix comprising the nanofiber web, although the invention also provides a nanocomposite wherein the active ingredient is loaded in, or adsorbed to, a vehicle comprising the nanofiber web.

A nanofiber web or mat for the purposes of this invention is a nonwoven randomly oriented or aligned collection of nanofibers. These nanofiber webs or mats are typically in the form of a thick and tangled mass defined by an open texture or porosity. For the purposes of this disclosure the terms nanofiber membrane, nanofiber web, nanofiber mat and nanofiber mesh are used interchangeably. The nanofiber web or mat is a membrane. Macroscopically, the membrane is a network of nanofibrous structure.

Nanofibers can be formed from various inorganic, organic, or biological polymers to form the nanofiber mat. Preferably these nanofibers are formed by electrospinning. However, other techniques such as drawing, template synthesis, phase separation or self-assembly may be used to produce nanofibers. All of these techniques are described in “An Introduction to Electrospinning and Nanofibers”, Ramakrishna et al., World Scientific, 2005. Nanofiber mats or webs can be modified by compression into pellets; by folding into homogeneous or heterogeneous layers; cutting into discs or rings; laminating onto carrier polymers, films, fabrics (woven or nonwoven), paper, or biological membranes; or chopped into short segments known as whiskers.

The nanofibers are preferably less than 3 micrometers in diameter, more preferably less than 500 nm in diameter, and most preferably less than 500 nm in diameter and greater than 2 nanometers in diameter. The thickness of the nanofiber web is less than 10 mm, more preferably less than 5 mm in thickness, and most preferably less than 1 mm in thickness. The weight of the active ingredient in the nanofiber web is less than 80 weight percent of the total weight of the active ingredient and the nanofiber web, more preferably less than 50 weight percent, and most preferably less than 20 weight percent.

Preferably, the polymers used to make the nanofibers need to be biocompatible. For the purposes of this patent, biocompatibility means the capability of coexistence with living tissues or organisms without causing harm by not being toxic, injurious, or physiologically reactive and not causing immunological rejection. The polymers used to make the nanofibers can be biodegradable or non-biodegradable and synthetic or natural. Examples of biocompatible, biodegradable synthetic polymers include but are not limited to polyesterurethane (Degrapol®), poly(ε-caprolactone), polydioxanone, poly(ethylene oxide), polyglycolide, poly(lactic acid) (PLA), poly(L-lactide-co-ε-caprolactone), poly(lactide-co-glycolide) (PLGA). One of the greatest potentials of electrospun fibers is in the field of tissue engineering. The natural polymers are biocompatible and have distinct advantages over synthetic polymers. These natural polymers include proteins (collagen, gelatin, fibrinogen, and silk, casein, chitosan) and polysaccharides (cellulose, hyaluronic acid). Preferably, the polymers are biodegradable and include polymers such as poly-(lactide) (PLA), poly (ε-caprolactone), polyethylene oxide, poly(L-lactide-co-ε-caprolactone) and poly-(lactide-co-glycolide) (PLGA). Non-biodegradable synthetic polymers such as nylon 4,6; nylon 6; nylon 6,6; nylon 12; polyacrylic acid; polyacrylonitrile; poly(benzimidazol (PBI); polycarbonate; poly(etherimide), PEI; poly(ethylene terephthalate); polymethylmethacrylate; polystyrene; polysulfone; poly(urethane); poly(urethane urea)s; poly(vinyl alcohol); poly(N-vinylcarazole); poly(vinyl chloride); poly(vinyl pyrrolidone); poly(vinylidene fluoride) (PVDF); and hydrogels such as galyfilcon and silicone hydrogels may be used alone or as co-polymers or laminates with other biodegradable or non-biodegradable polymers. Such non-biodegradable polymers or copolymer blends may be used, for example, as a carrier for drug delivery, for glaucoma surgical adjuncts, orbital/paranasal sinus surgical repair, orbital repair after enucleation, or tissue engineering purposes. It may be necessary to polymerize two different homopolymers to form a copolymer (random or block) or by physical mixing of two or more polymers to form a polymer blend.

In a preferred embodiment, PLGA is the polymer used to produce the nanofiber web or mat, since it degrades harmlessly to lactic and glycolic acids in vivo, which are then metabolized by cells.

Electrospinning or encapsulation techniques similarly allow for sustained drug release from the PLGA polymer carrier. PLGA has been successfully electrospun with many drugs, from tetracycline to the non-steroidal pain reliever ibuprofen³². The formulation and characteristics of the drug-PLGA matrices is influenced not only by the polymer used to produce the nanofiber web or mat, but also by the type of drug chosen for binding. A 20% concentration of ibuprofen in 50:50 poly (lactide-co-glycolide), for example, will have a different release profile from a 20% concentration of corticosterone in the same polymer.³³

In one embodiment of this invention, the nanofiber mat is formed in to a pledget to be soaked in solutions of active ingredients for use as a DDD placed in the conjunctival fornices for treatment of ocular diseases. For example, a nanofiber mat of biocompatible polymeric material in the form of a pledget may be soaked in drug solutions (such as antibiotics, non-steroidal anti-inflammatory drugs, mydriatic and cycloplegic drugs), for use as a DDD in the conjunctival fornices for dilation of the iris before surgical/laser procedures or ophthalmological examinations.

An “active ingredient” for the purposes of this invention is defined as any material that can be introduced in to the body. Active ingredients include medicinal agents and biological drugs. As defined by the National Cancer Institute, a “biological drug” is a substance that is made from a living organism or its products and is used in the prevention, diagnosis, or treatment of cancer and other diseases. Such biological drugs include antibodies, interleukins, growth factors, and vaccines. A biological drug may also be called a biologic agent or a biological agent.

By the term “medicinal agent” is intended any substance or mixture of substances which may have any clinical use in medicine. Thus medicinal agents include drugs, enzymes, proteins, peptides, glycoproteins, immunoglobulins, nucleotides, RNA, siRNA, DNA, hormones or diagnostic agents such as releasable dyes or tracers which may have no biological activity per se, but are useful for diagnostic testing, e.g., MRI.

Examples of classes of medicinal agents that can be used in accordance with the present invention include antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunosuppressants, gastrointestinal drugs, diuretics, corticosteroids and enzymes. It is also intended that combinations of medicinal agents can be used in accordance with the present invention. Thus, in one embodiment of the present invention focal delivery and application of a medicinal agent to tissue is achieved. Focal application can be more desirable than general systemic application in some cases, e.g., chemotherapy for localized tumors, because it produces fewer side effects in distant tissues or organs and also concentrates therapy at intended sites. Focal application of growth factors, anti-inflammatory agents, immune system suppressants and/or antimicrobials by the membranes of the present invention is an ideal drug delivery system to speed healing of a wound or incision.

Drugs may include but are not limited to many classes such as Anti-infectives, Antibiotics, Antituberculosis agents, Anti-fungal agents, Anti-viral agents, Anti-parasitic agents, Anti-rheumatic agents, Non-steroidal anti-inflammatory drugs (NSAID), Corticosteroids, Immunomodulators, Biologicals, Anti-neoplastic agents and others.

Examples of Antibiotics are aminoglycosides, beta-lactam antibiotics, miscellaneous antibiotics (e.g., clindamycin, vancomycin, oxazoladinones). Examples of Anti-fungal agents are amphotericin B, fluconazole, among others. Examples of Anti-viral agents are anti-HIV agents and other antivirals. Examples of Anti-parasitic agents are amebicides, anti-helminthics. Examples of Anti-rheumatic agents are Salicylates, e.g., acetylsalicylates and others.

Non-steroidal anti-inflammatory drugs (NSAID) are for example acetylsalicylic acid, naproxyn sodium, ibuprofen, diclofenac, indomethacin, cyclooxygenase-2 (COX-2) inhibitors (e.g., rofecoxib) and others. Corticosteroids(Glucocorticoids) are for example Betamethasone, budesonide, cortisone, decadron, dexamethasone, fluocinolone, fluticasone, loteprednol etabonate, methylprednisone, prednisone, prednisolone acetate, prednisolone phosphate, rimexolone, triamcinolone acetonide, Immunomodulators, Azathioprine, mycophenylate mofetil, cyclophosphamide, cyclosporine A, rapamycin, tacrolimus, Methotrexate and others. Biologicals are for example anti-bodies such as, tumor necrosis factor (TNF) blockers (such as adalimumab, infliximab, etanercept), daclizumab, aptamers, growth factors, peptides, nucleotides such as DNA, RNA, siRNA and others. Included are compounds which promote healing and re-endothelialization such as VEGF, Estradiols, antibodies, NO donors, and BCP671. Anti-neoplastic agents are drugs used for treatment of primary central nervous system lymphoma, ocular melanoma and retinoblastoma.

The preferred medicinal agents are corticosteroids, immunomodulators, and biologicals such as aptamers, monoclonal antibodies, and nucleotides. The preferred corticosteroids are budesonide, decadron, dexamethasone, fluocinolone, fluticasone, loteprednol etabonate, methylprednisone, prednisone, prednisolone acetate and phosphate, rimexolone and triamcinolone acetonide. The preferred immunomodulators are azathioprine, mycophenylate mofetil, cyclophosphamide, cyclosporine A, rapamycin, tacrolimus, and methotrexate. The preferred monoclonal antibodies are TNF blockers, such as adalimumab, infliximab and etanercept, daclizumab, and anti-VEGF agents such as ranibizumab, bevacizumab, and aptamers.

A bodily fluid for the purposes of this invention is any fluid found in the body of humans and animals including intra- and extracellular fluids. Examples of these extracellular fluids are subcutaneous fluids, enteral fluids, parenteral fluids, peritoneal fluids, blood, cerebrospinal fluids, glandular fluids such as pancreatic, hepatic, gallbladder, plasma and ocular fluids.

The preferred bodily fluid is an ocular fluid. Ocular fluids are for example vitreous humor, aqueous humor, tears, and the extracellular fluid found in potential spaces such as the subconjunctival and sub-tenon's spaces.

Vitreous humor/vitreous cavity. The vitreous cavity is the space from the lens to the retina, filled with a jelly-like substance called vitreous. The vitreous body occupies the major volume of the globe of the eye (approximately 4.5 ml in humans), serving as a shock-absorbing gel by absorbing and redistributing forces applied to surrounding ocular tissues while remaining transparent for transmission of light. The physical attributes of the vitreous gel derive from the collagen framework and the dispersed hyaluronic acid, which accounts for the viscosity.

Composition: The vitreous gel contains 98% water and 0.1% colloids.

Aqueous humor/anterior and posterior segments of the eye. The anterior segment of the eye is bounded by the inner layer of the cornea (endothelium), posteriorly by the plane of the iris, and laterally by the angle formed by the cornea and iris. The posterior segment is the small space between the back surface of the iris and the lens. The aqueous humor is a watery fluid produced by the ciliary body processes in the posterior segment, flowing anteriorly through the pupil and circulating in the anterior chamber by convection currents, before exiting the eye through the trabecular meshwork. Aqueous humor is a thin, clear fluid containing electrolytes, oxygen, and a small amount of protein to bath and nourish the lens and inner cornea.

Tear film/conjunctival fornices and corneal. The tear film is a watery fluid secreted by the main and accessory lacrimal glands to lubricate and moisten the cornea to maintain a clear medium through which to transmit light.

Extracellular fluid. The spaces between the conjunctiva and tenon's capsule, between tenon's capsule and sclera, and retroorbital spaces for example, have extracellular (ECF) fluid bathing the tissues. This extracellular fluid can be divided into interstitial fluid and blood plasma in mammals. ECF contains cations, anions, glucose, and low levels of proteins. Drugs delivered into the extracellular fluid of these spaces may be absorbed or transported into cells for therapeutic effects.

The drug delivery composition of this invention may be administered in a number of ways. In general the nanofiber web containing the active ingredient is introduced into the bodily fluid and the active ingredient is allowed to release into the fluid in a controlled manner over a period of time. In the case of an ocular fluid, the nanofiber web needs to be positioned or placed in such a manner so as to minimally impair the vision. Preferably, the vision is not affected at all as in most of the applications described below. However, there may be transient impairment of vision due to the semi-transparent characteristics of the corneal shield. Visual impairment is conventionally described as a diminution of the Snellen visual acuity and/or a decrease in peripheral field on visual field testing. By minimal impairment of vision, we mean less than 2 line decrease in Snellen acuity and/or a decrease in 15 degrees of peripheral field by formal visual field testing.

The drug delivery composition containing the nanofiber web, and the active ingredient may be used in the following methods.

1. Corneal shield. A nanofiber mat is fashioned into a contact lens-shaped device (shield) for protection of the cornea and conjunctiva as well as drug delivery. The corneal shield will comprise a biocompatible and biodegradable polymer such as electrospun collagen. These nanofiber shields will be hydrated in saline or medicinal agent for treatment and prophylaxis of infections, pain relief, and to promote wound healing. The ocular fluid in this case will be tears.
2. Forniceal pledgets. Nanofiber mats are formed of biocompatible polymers, which may be biodegradable or non-biodegradable polymers for delivery of medical agents. These will be manufactured with drugs or hydrated with drugs and placed in the superior or inferior conjunctival fornices. The primary uses would be for pre-operative mydriasis, the treatment of dry eye (keratoconjunctivitis sicca), intraocular infection or inflammation, and glaucoma. The ocular fluid would be tears.
3. Subconjunctival drug delivery. An entry will be made into the subconjunctival space by incision or needle for placement of biocompatible biodegradable and non-biodegradable nanofiber materials. These nanofiber mats will be used for delivery of medicinal agents to treat inflammation, infection, etc. The bodily fluid would be extracellular fluid.
4. Sub-tenon's drug delivery. An incision or needle will be used to enter the sub-tenon's space for delivery of medicinal agents to the episcleral and intravitreal spaces (via trans-scleral movement). Examples of use would be to insert nanofiber mats, discs, or pellets for infection, inflammation, and treatment of ocular tumors. The bodily fluid would be extracellular fluid.
5. Intravitreal drug delivery. For intravitreal drug delivery, a nanofiber pledget or pellet will be inserted into the vitreous cavity via a pars plana approach. For human use, a 1 mm×6 mm pledget of drug-nanofiber mat will be inserted 4 mm posterior to the limbus in phakic eyes and 3.5 mm posterior to the limbus in aphakic eyes. A triangular flap of conjunctiva is reflected in the inferior globe, exposing tenon's capsule. A similar triangular flap of tenon's capsule is created, down to bare sclera. Using a microvitreoretinal blade (MVR), a perforating sclerotomy is created with the tip of the blade directed toward the optic nerve. Next the nanofiber-drug pledget is placed directly over the sclerotomy. Using the tip of the MVR blade, the pledget is inserted into the vitreous cavity by folding the pledget at the midpoint. A single 10-0 nylon “x” suture is used to close the sclerostomy. Tenon's capsule and conjunctiva are teased into place. The bodily fluid would comprise vitreous gel.
6. Anterior chamber drug delivery. For anterior chamber drug delivery, nanofiber pellets would be inserted at the time of anterior segment surgery by incision or in the outpatient setting via paracentesis. These nanofiber mats or pellets will be used for delivery of medicinal agents to treat inflammation, infection, opacification of the posterior capsule, etc. The bodily fluid would be aqueous fluid.
7. Scaffolding. The 3 dimensional characteristics of nanofibers, particularly the interconnected pores, lens themselves to tissue regeneration end uses. Incorporation of growth factors and progenitor cells can be used to supply cells to damaged or degenerated tissues. For example, corneal stem cells can be embedded into nanofiber webs and sutured into position to resupply resected or damaged corneal and conjunctival tissues. The bodily fluid would be tears. Retinal and retinal pigment epithelial cells could similarly be placed into the eye to recover lost function. Scaffolds would be placed either within the vitreous gel or subretinally in extracellular fluid. Nanofibers can be used to replace the globe after enucleation since they allow tissue growth into implant, which decreases the likelihood of extrusion. The orbit would be the location and bodily fluids would include blood and extracellular fluid.
8. Non-biodegradable nanofiber mats will be placed under the conjunctiva or tenon's capsule to act as synthetic reinforcing grafts after glaucoma surgery, or trauma, which would promote cellular ingrowth and prevent extrusion of implants. These reinforcing grafts would be used to treat scleral thinning in scleral disorders. Non-biodegradable nanofiber mats would be used as glaucoma setons as a means of facilitating outflow in glaucoma filtration surgery.

Nanofiber web: a non-woven, porous mesh of fibers with diameters in the 1-1000 nanometer range, and lengths from Electrospinning

The following nonlimiting examples are provided to exemplify the invention.

### Example 1

I. Method of Manufacture: The aspirin (acetylsalicylic acid, ASA) was incorporated into polyurethane in 3 different concentrations: 1%, 5%, and 10% ASA by mixing polyurethane (PU) and respective w/w concentrations in N,N-dimethylformamide (DMF) solvent. The mixed polymer solution was injected via a syringe pump and electrospun onto a grounded drum under high dc voltage under usual conditions.

The method of drug incorporation was similar to the incorporation of itraconazole and ketanserin into segmented polyurethane as detailed in the article by G. Verreck et al., “Incorporation of drugs in an amorphous state into electrospun nanofibers composed of a water-insoluble, non-biodegradable polymer.” J Controlled Release. Vol 92, 3, 30 Oct. 2003, 349-360.¹⁻⁴

### Example 2

The corticosteroid triamcinolone acetonide (TA) was incorporated into the biodegradable polymer poly (lactide-co-glycolide) [PLGA] by electrospinning as described in Example 1. A polymer solution of 0.11% TA was mixed with 9.01 gm PLGA in 2 ml tetramethylfuran (TMF) and 15 ml DMF (˜36.25% polymer). The polymer-drug solution was injected via a syringe pump and electrostatically spun at 16 and 24 kV. The formed nanofibers were collected as a non-woven fabric.
1. Verreck G, Chun I, Peeters J, Rosenblatt J, Brewster M E. Preparation and characterization of nanofibers containing amorphous drug dispersions generated by electrostatic spinning. Pharm Res. May 2003; 20(5):810-817.
2. Brewster M E, Verreck G, Chun I, et al. The use of polymer-based electrospun nanofibers containing amorphous drug dispersions for the delivery of poorly water-soluble pharmaceuticals. Pharmazie. May 2004; 59(5):387-391.
3. Verreck G, Chun I, Rosenblatt J, et al. Incorporation of drugs in an amorphous state into electrospun nanofibers composed of a water-insoluble, nonbiodegradable polymer. J Control Release. Oct. 30, 2003; 92(3):349-360.
4. Xie J, Wang C H. Electrospun micro- and nanofibers for sustained delivery of paclitaxel to treat C6 glioma in vitro. Pharm Res. August 2006; 23(8):1817-1826.

II. Drug elution. Preliminary results of drug loading of ASA into PU demonstrated a burst release of the water-soluble ASA. Demonstration of sustained release of TA from the biodegradable polymer PLGA will be performed with timed assays by Ultraviolet-visible light spectroscopy. After assessing nanofiber mat uniformity, multiple round samples will be punched from the TA-PLGA nanofiber mat using a 6 mm metal punch. The 0.25 mm thin membranes will be stripped from their paper backing with jeweler's forceps and placed in closed-centrifuge vials. A 2 ml aliquot of phoshate-buffered saline (PBS) will be pipetted into the tubes with an adjustable Thermo™ volumetric pipette, totally immersing the samples. Triplicate samples of the drug polymer concentrations will be analyzed for timepoints 0, 24 hrs, 48 hrs, 72 hrs, 96 hrs and at weekly intervals for 6 months. At time 0, PBS will be immediately removed for analysis from the appropriate vial, leaving the nanofiber sample. Fresh PBS (2 ml) will be added back to the tube. All other timepoint samples will be incubated at 37° C. in a water-jacketed Napco incubator with periodic vortexing. At the designated sample times, the PBS will be removed and replaced with fresh 2 ml aliquots of PBS. The extractant from each timepoint will be refrigerated prior to analysis by high performance liquid chromatography (HPLC) and/or immunologic methods such as enzyme-linked immusorbent assay (ELISA). A sustained release of TA over a period of four to six months will be obtained using the procedure above.

III. Method of Delivery. For intravitreal drug delivery, a nanofiber pledget will be inserted into the vitreous cavity via a pas plana approach. For human use, a 2 mm×6 mm pledget of drug-nanofiber mat will be inserted 4 mm posterior to the limbus in phakic eyes and 3.5 mm posterior to the limbus in aphakic eyes. A triangular flap of conjunctiva is reflected in the inferior globe, exposing tenon's capsule. A similar triangular flap of tenon's capsule is created, down to bare sclera. Using a microvitreoretinal blade (MVR), a perforating sclerotomy is created with the tip of the blade directed toward the optic nerve. Next the nanofiber-drug pledget is placed directly over the sclerotomy. Using the tip of the MVR blade, the pledget is inserted into the vitreous cavity by folding the pledget at the midpoint. A single 10-0 nylon “x” suture is used to close the sclerostomy. Any vitreous wicks will be severed with Wescott scissors. Tenon's capsule and conjunctiva are teased back into place and sutured into position.

Similarly, surgical delivery of nanofiber mats may be improved by folding and/or compressing the nanofiber mats into pellets which may be injected into the vitreous cavity via the pars plana.

We claim:

1. A drug delivery composition comprising:
a nanofiber web;
an active ingredient; and
a bodily fluid.

2. A drug delivery composition according to claim 1, wherein the bodily fluid is an ocular fluid.

3. A drug delivery composition according to claim 2, wherein the ocular fluid is selected from the group consisting of vitreous humor, aqueous humor, tears, and extracellular fluid.

4. A drug delivery composition according to claim 1, wherein the active ingredient is selected from a group consisting of medicinal agents and bioactive materials.

5. A drug delivery composition according to claim 4, wherein the medicinal agent is a drug.

6. A drug delivery composition according to claim 5, wherein the drug is selected from the group consisting of corticosteroids, immunomodulators and monoclonal antibodies.

7. A drug delivery composition according to claim 6, wherein the corticosteroids is selected from the group consisting of budesonide, decadron, dexamethasone, fluocinolone, fluticasone, loteprednol etabonate, methylprednisone, prednisone, prednisolone acetate and phosphate, rimexolone and triamcinolone acetonide.

8. A drug delivery composition according to claim 4, wherein the bioactive materials are selected from the group consisting of growth factors, nutraceuticals and antimicrobials.

9. A drug delivery composition according to claim 1, wherein the nanofiber web comprises of a biodegradable polymer.

10. A drug delivery composition according to claim 1, wherein the nanofiber web comprises of a biocompatible polymer.

11. A drug delivery composition according to claim 9, wherein the polymer is selected from the group consisting of poly-(lactide) (PLA), polycaprolactone, poly-(vinyl alcohol), biodegradable polyester, chitosan, poly-(propylene carbonate), and poly-(lactide-glycolide).

12. A drug delivery composition according to claim 1, wherein the thickness of the nanofiber web is less than 5 mm.

13. A drug delivery composition according to claim 1, wherein the nanofiber web is selected from a form consisting of a pledget, whiskers, pellet, contact lens, shield, and disc.

14. A method of drug delivery comprising:
positioning a nanofiber web containing an active ingredient into a bodily fluid.

15. The method of drug delivery according to claim 14, wherein the bodily fluid is an ocular fluid.

16. The method of drug delivery according to claim 15, wherein the nanofiber web is placed within the ocular fluid so that vision is minimally impaired.

17. The method of drug delivery according to claim 15, wherein the ocular fluid is selected from the group consisting of vitreous humor, aqueous humor, tears, and extracellular fluid.

18. The method of drug delivery according to claim 14, wherein the active ingredient is a drug selected from the group consisting of corticosteroids, immunomodulators and biologicals such as aptamers, monoclonal antibodies, and nucleotides.

19. The method of drug delivery according to claim 18, wherein the corticosteroids is selected from the group consisting of budesonide, decadron, dexamethasone, fluocinolone, fluticasone, loteprednol etabonate, methylprednisone, prednisone, prednisolone acetate and phosphate, rimexolone and triamcinolone acetonide.

20. The method of drug delivery according to claim 14, wherein the nanofiber web comprises of a biocompatible polymer.

21. The method of drug delivery according to claim 14, wherein the nanofiber web is selected from a form consisting of a pledget, whiskers, pellet, contact lens, shield, and disc.
